(19) United States
(12) Patent Application Publication
(10) Pub. No.: US 2009/0275656 A1
(43) Pub. Date: Nov. 5, 2009

(54) Medicinal Compositions of Salts, Chelates and/or Free Acids of Alpha Hydroxyl Organic Acids and Related Processes and Methods

(76) Inventor: Ronald W. Pero, Sandgate, VT (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 11/886,345

(22) PCT Filed: Mar. 16, 2006
(86) PCT No.: PCT/US2006/009394

§ 371 Date: Sep. 14, 2007

Related U.S. Application Data
(60) Provisional application No. 60/662,446, filed on Mar. 16, 2005.

(51) Int. Cl. A61K 31/19; C07C 51/00; C07C 51/347; A61P 37/00; A61P 35/00; A61P 29/00

(52) U.S. Cl. 514/557; 562/579; 562/508; 562/584; 549/315; 562/582; 562/589; 562/585; 562/471

(57) ABSTRACT

Processes are described for the production of an isolated medicinal composition for administration to mammals which comprises an effective amount of a free acid, salt or chelate of at least one naturally occurring form of an alpha hydroxyl organic acid comprising combining at least one naturally occurring form of an alpha hydroxy organic acid with an amount of base in an aqueous solution, wherein a total content of alpha hydroxyl organic acids amounts to between about 0.5% and about 35% w/w of the solution, for a time to hydrolyse substantially all forms of alpha hydroxyl organic acids present, neutralizing the solution to a pH between about 6.9 and about 7.6 to yield a free acid, salt or chelate of free acids of substantially all naturally occurring forms of alpha hydroxyl organic acids, and optionally lyophilizing the solution to produce an isolated medicinal composition. Compositions produced by these processes are disclosed and claimed. Method are described of enhancing DNA repair, enhancing an immune response, controlling inflammation, or inhibiting the progress of a tumor, comprising administering an effective amount of a composition produced by the processes described herein.

Priority is indicated from U.S. Provisional Application Ser. No. 60/662,446, filed Mar. 16, 2005, which is herein incorporated in its entirety by reference.

### FIELD OF THE INVENTION

The present invention is directed to processes to convert substantially all forms of naturally occurring alpha hydroxyl organic acids into each corresponding free acid, salt or chelate, for example, quinic acid in plant material into a chelate form, and to the related production of improved medicinal compositions which exhibit increased biological efficacy and decrease toxicity wherein each corresponding free acid, salt or chelate are the forms that are substantially present.

### BACKGROUND OF THE INVENTION

Uncaria tomentosa and other “Cat's Claw” species belong to the family Rubiaceae, subfamily cinchonoideae and genus Uncaria. These type of plants grow in tropical regions and consist of climbing woody vines with hook-like thorns. The two primary species from historical medical practice are Uncaria tomentosa and Uncaria guianensis. Historically there are two main chemical classes of compounds found coming from this family of plants. They are organic acids occurring naturally as free acids, salts or esters such as quinic acid in chinchona bark first described in 1932, or alkaloids in chinchona bark such as quinine extracted in 1952. Hence it is not surprising that alkaloids and organic acid analogs are found quite commonly within the Uncaria species.

The primary historic medicinal practice with Cat's Claw extracts have evolved from heating bark or other plan parts to near boiling temperatures on an open fire, while the bark is covered in water for an overnight period. The next morning the partially evaporated extract is normally drank as a tea.

Cat's Claw is a plant well-known to have thousands of years of use as a historical traditional medicine, where it is indigenous to South America. The Ashinka Indians prepared a concoction from boiling the bark in water on an open fire overnight before decanting the mixture free from plant parts and sipping the resultant extract daily to help control infections, inflammatory disorders and even mental state. A recent publication reports that Uncaria tomentosa (cat's claw) extract protects mice against ozone-induced lung inflammation. (Cisneros, F. J., et al., J. Ethnopharmacol., 15; 96(3):355 (January 2005).

### Esters/CAEs

Commercial preparations of Cat's Claw products have been focused into standardizing formulations based on either alkaloid content or carboxy alkyl ester (CAE) content. U.S. Pat. No. 6,964,784, issued Nov. 15, 2005, is drawn toward the isolation, purification and identification of the biologically active components of previously known uncaria extracts. Particularly identified as the biologically active components of uncaria extracts are quinic acid complexes including quinic acid esters, previously generically identified as carboxy alkyl esters (CAEs) that contain quinic acid. The isolated bioactive components are identified as quinic acid ester analogs, preferably quinic acid lactone.

Sheng, Y., et al., similarly published a report entitled, An Active Ingredient of Cat's Claw Water Extracts Identification and Efficacy Of Quinic Acid, wherein the active ingredients of uncaria were chemically defined as quinic acid, i.e., the free acid, per se, as well as quinic acid esters that were included earlier in a generic description as carboxy alkyl esters (CAEs). J. Ethnopharmacol., 15; 96(3):577 (January 2005).

### Free Acid and Salts

U.S. application publication No. 20050176825, filed Oct. 21, 2004, published Aug. 11, 2005, is directed to the isolation, purification and structural identification of the bioactive component of water extracts of uncaria. The disclosure acknowledges that although the bioactive component has previously been identified as quinic acid lactone and other related quinic acid esters the bioactive component is elucidated as quinic acid and quinic acid salts, per se, including ammonia treated quinic acid. Ammonia chelates are, however, merely identified as artifacts which could fundamentally exist in a de minimus amount incidental to the creation of ammonium salts.

Akesson, C., et al., published a report entitled, Quinic Acid is a Biologically Active Component of the Uncaria Tomentosa Extract C-Med 100, wherein quinic acid is identified a key bioactive component of the uncaria extract, although the authors acknowledge that it probably does not occur naturally in the free acid form. It is hypothesized that a novel salt, chelate or hydrolyzable ester are more favorably indicated rather than a simple ammonium salt. Moreover, the authors point out that the content of quinic acid equivalents in the form of esters, chelates or salts could contribute significantly to the in vivo biological effect. Int. Immunopharmacol., 5(1):219 (2005).

Both Cat's Claw extracts and quinic acid have been shown previously to reduce beta amyloid plaque associated with neurodegeneratice diseases such as Alzheimers. U.S. Pat. No. 6,346,280 and U.S. application publication Nos. 20010055630 and 20010047032. These disclosures, however, are confined to beta amyloid bodies and neurodegenerative diseases and did not include any general reference to immune, anti-inflammatory, anti-tumor and DNA repair processes.

However, as recently published by scientists of the U.S. Food and Drug Administration (FDA), “it appears that the presence of unknown substances has an important role in the overall effects of cat's claw [uncaria] extracts is an important factor for consideration”. Valerio, L G, et al., Toxicological Aspects of the South American Herbs Cat's Claw (Uncaria tomentosa), Toxicol. Rev., 24(1): 11 (2005).

### SUMMARY OF THE INVENTION

The present invention is directed to compositions of bioactive components of alpha hydroxy organic acids that occur naturally as free acids or salts or chelates or esters such as quinic acid and that can inhibit NF-kB activation in human Jurkatt T cells to least 50% of the maximum in vitro response at a dose of 1.25 mg/ml or lower and/or that can cause growth arrest of spleen cells cultured in vitro in the presence of mitogen (Con A) at a dose of 2 mg/ml or lower and/or that upon systemic administration in a sufficient amount for treatment enhance immune, anti-inflammatory, anti-tumor, or DNA repair

The invention is further directed to processes for the conversion of plant extracts containing alpha hydroxy organic acids that are present as free acids, or salts or chelates or esters and that can be converted to either the free acid or salt or chelated forms by treatment of said extract with strong base, for example, about 1M NaOH or less than 10% ammonia (e.g., between about 0.8% and 10%) or both for about 2 hours (between about 15 minutes and about 4 hours) and that contain ≧ about 0.5, % (gm/100 gm) of the free organic acid or salt or chelate forms after strong base treatment of said extract; for example quinic acid; and that can inhibit NF-kB activation in Jurkatt T cells to least 50% of the maximum in vitro response at a dose of 1.25 mg/ml or lower; or that can cause growth arrest of spleen cells cultured in vitro in the presence of mitogen (Con A) at a dose of 2 mg/ml or lower; and that systemic treatment is in a sufficient amount to enhance immune, anti-inflammatory, anti-tumor, or DNA Repair

In addition, the current invention is directed to quinic acid per se in an efficacious pharmaceutical composition for administration or the compound in its other natural occurring forms of salts, chelates or esters and that is limited to only being produced outside the bodies of warm blooded animals via the shikimate pathway; and that can enhance the bodily process of DNA repair, thereby increasing the removal of DNA damage and the health associated consequences thereof; and that is broadly present in food sources in small amounts; and that it is essential for maintenance of DNA repair and anti-aging good health—now identified as vitamin DNA essential for maintenance of good health to prevent aging.

The invention is also directed to an edible food or plant composition containing quinic acid, its salts or chelates in an amount ≧0.5 gm per 100 gm serving so that after human consumption by eating or drinking an efficacious dose of ≧1 mg/kg is provided within 24 hours to be sufficient to enhance immune, anti-inflammatory, anti-tumor, or DNA repair processes.

The invention is further directed to a composition produced by a process of the present invention or compositions containing ≧0.5% w/w of quinic acid or its salts or all said compositions that has been lyophilized and exhibits at least one property from the group consisting of: (a) inhibits NF-kB activation in human Jurkatt T cells to at least 50% of the maximum in vitro response at a dose of 1.25 mg/ml or lower, (b) causes growth arrest of spleen cells cultured in vitro in the presence of mitogen (Con A) at a dose of 2 mg/ml or lower, and (c) systemic administration to a mammal at doses between 1 mg/kg and 200 mg/kg to enhance immune, anti-inflammatory, anti-tumor, or DNA repair

The present invention is directed to processes for the production of an isolated medicinal compositions for administration to mammals which comprise an effective amount, e.g., a dosage form to effect the delivery of between about 0.2 mg to about 10 mg/kg in a human, of a free acid, salt or chelate of at least one naturally occurring form of an alpha hydroxyl organic acid.

In addition, the current invention is directed to processes for the production of an isolated medicinal compositions from aqueous extracts of plant material for administration to mammals which comprise an effective amount of a free acid, salt or chelate of at least one naturally occurring form of an alpha hydroxyl organic acid.

The invention is further directed to a process for the production of an isolated medicinal composition from substantially pure quinic acid for administration to a mammal which comprises an effective amount of a quinic acid chelate, preferably an ammonium chelate in about a 1:1.54 ratio of quinic acid to ammonium ion.

Further the invention is directed to a process for the production of a functional food comprising hydrolysing substantially all forms of alpha hydroxyl organic acid esters present in the food to yield a salt and/or chelate of free acids of substantially all naturally occurring forms of alpha hydroxyl organic acids in the food.

The invention is also directed to products produced by the processes of the invention.

The invention is particularly directed to compositions produced by the processes of the invention containing free acid, salts or chelates which a) inhibit NF-kB activation in Jurkatt T cells to least 50% of the maximum in vitro response at a dose of 1.25 mg/ml or lower, and/or b) causes growth arrest of spleen cells cultured in vitro in the presence of mitogen (Con A) at a dose of 2 mg/ml or lower, and/or c) enhances immune, anti-inflammatory, anti-tumor, DNA repair or tryptophan uptake processes when an effective amount is administered to a mammal.

In addition, the current invention is directed to a method of enhancing DNA repair, enhancing tryptophan uptake, enhancing an immune response, controlling inflammation, or inhibiting the progress of a tumor, comprising administering an effective amount of a composition produced by a process of the invention.

### BRIEF DESCRIPTION OF THE FIGURES

FIG. 1 illustrates in vitro growth inhibition expressed as IC₅₀ values (i.e. the dose that inhibits 50% of growth) induced by various quinic acid salts, chelates or salts/chelates (molecular structure at saturation undetermined) in cultured HL-60 cells. QA-H+=free quinic acid (H+), QA-NH₄.+=quinic acid ammonium chelate, QA-Na+=quinic acid sodium salt, QA-K+=quinic acid potaasium chelate, QA-Zn++=quinic acid zinc salt/chelate, QA-Li+=quinic acid lithium salt/chelate, Ca++=quinic acid calcium chelate, quinic acid histidine salt/chaelate=QA-histidine, quinic acid lysine salt/chelate=QA-lysine, and C-Med-100=non pH adjusted water extract.

FIG. 2 shows growth arrest of C57BL/6 mouse spleen cells induced by Garcinia extracts and compared to the Cat's Claw water extract, C-Med-100. The Garcinia extracts were Citrimix and nutrated Citrimix (Nu-Citrimix) which was prepared by first conversion to the free acid with 0.5 M HCl, freeze dried and then neutralized with 5 M ammonia to pH=7.5 creating citrimix ammonia chelate (Table 1). Growth of spleen cells was evaluated in response to the mitogen Con A (2.5 μg/ml) after 48 hours by radioactive thymidine incorporation. Comparison is made at doses in mg/ml for inhibiting 25%, 50% and 90% of the growth of spleen cells; i.e. IC dose values.

FIG. 3 demonstrates growth arrest of C57BL/6 mouse spleen cells induced by quinic acid (H+), quinic acid NH₄+ chelate and NH₄Cl. Quinic acid NH₄+ chelate was synthesized by neutralizing to pH=7.5 quinic acid (H+) with ammonium hydroxide. The amount of ammonium hydroxide necessary to neutralize quinic acid (H+) was also neutralized with HCl to create an NH₄Cl control. Growth of spleen cells was evaluated in response to the mitogen Con A (2.5 μg/ml) after 48 hours by radioactive thymidine incorporation. Comparison is made at concentrations in mg/ml for inhibiting 25%, 50% and 90% of the growth of spleen cells; IC values.

FIG. 4 illustrates the evaluation of NF-kB in Jurkat T cells as previously described in detail after treatment with quinic acid (H+), quinic aid ammonium chelate neutralized to pH=7.5 or the equivalent amount of ammonium hydroxide (NH₄Cl).

FIG. 5 shows growth arrest of C57BL/6 mouse spleen cells induced by the Cat's Claw water extracts, C-Med-100 or Nu-CC 100. Nu-CC 100 has been nutrated by treatment of C-Med-100 with 1% ammonia and then freeze dried to form chelates of resident organic acid analogs. Growth arrest of C57BL/6 mouse spleen cells induced by the Cat's Claw water extracts, C-Med-100 or Nu-CC100 was evaluated in response to the mitogen Con A (2.5 μg/ml) after 48 hours by tritiated thymidine incorporation. Nu-CC100 has been nutrated by treatment of C-Med-100 with 1% ammonia and then freeze dried. Comparison is made at doses in mg/ml for inhibiting 25%, 50% and 90% of the growth of spleen cells; IC values.

FIG. 6 illustrates the detection of hippuric acid after oral administration of 6 gm Quinmax™, the ammonia chelate of QA.

FIG. 7 shows the structural similarity between hippuric acid and kynurenine and it is a key intermediate in the tryptophan degradation pathway. Hippuric acid metabolized from QA may competitively inhibit tryptophan degradation by chemical binding to the kynurenine enzyme substrate site.

FIG. 8 demonstrates DNA Damage Recovery in the Rat Induced by C-Med-100 (80/kg), Quinic acid (200 mg/kg), or ammonia treated quinic acid (200 mg/kg) after DXR (2 mg/ml) Treatment. White blood cell (WBC) growth after 15 days estimated as 10¹²WBC/liter blood. DXR=doxorubicin DNA damaging agent.

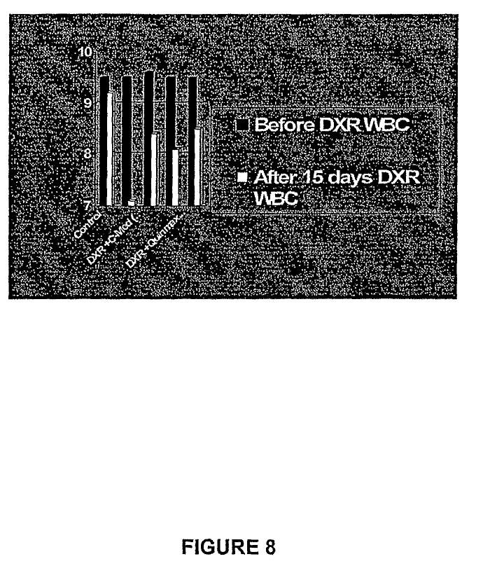

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

The term “alpha hydroxy organic acid”, as used herein, refers to the location of a hydroxyl (—OH) group alpha to (or the carbon next to) the carbon containing a carboxyl (—COOH) group. The term “all forms of alpha hydroxy organic acid” as used herein refers to esterified alpha hydroxy organic acids, free alpha hydroxy organic acids (H+) and its salts and chelates.

Here it is disclosed that ammonium ions, for example, both enhance the nutritive value and the efficacious modes of action of alpha hydroxy organic acid bioactive components present in the plant extracts.

Quinic acid (QA) is a polyhydroxylated alpha hydroxy monocarboxylic acid, and so can form chelates as well as salts and esters. Hence, it is disclosed here that indeed quinic acid formed salts when neutralized with some bases like sodium hydroxide, and chelates when neutralized with other bases like potassium or ammonium hydroxides. See, e.g., Table 1, infra.

The term “all forms of quinic acid” as used herein refers to free quinic acid (H+), quinic acid salts, quinic acid chelates or esters of all natural occurring esters of quinic acid for example the carbohydrate esters in cats claw, tannins or chlorogenic acid. An ester of quinic acid is defined as a compound yielding quinic acid after treatment with strong base or alkali.

A carboxy organic acid salt is defined by the fact that for every mole of carboxyl group it takes one corresponding mole of base to neutralize it. Hence, a 1:1 molar salt of a monocarboxy organic acid is a true salt, a 2:1 molar salt if a dicarboxy acid is being neutralized. Carboxy organic acids such as quinic acid and alpha hydroxyl citric acid do not achieve theoretical molar ratios at saturated molecular equilibrium indicating stable chelated forms to be present.

As used herein. the term “chelate” refers to a ratio of free acid to ion (e.g, ammonium ion) wherein the represented ion in the ratio is not a whole number, e.g., 1:1.2, 1:1.3, 1:1.4, 1:1.5 and 1:1.6, as well as values in between, e.g., 1:1.54 (quinic acid saturated with ammonium ions). However, depending upon the conditions, particularly the pH of the solution, the chelate ratios vary. Although as discussed herein close to a 1:1.54 ratio is preferred quinic acid chelate compositions of the present invention are by no means limited to those with a 1:1.54 ration of quinic acid to ammonium ion.

TABLE 1
Determination of salts and chelates of some naturally occurring
polyhydroxylated and polycarboxylated organic acids.

| | Molar ratio of | Experimental molar ratio | Determination of |
|---|---|---|---|
| | salt theoretical | of molecular equilibrium | structure existing |
| Organic acid salt | carboxy(−)/cation(+) | in water | in water |
| Quinic acid sodium salt | 1:1 | 1:1 | salt |
| Quinic acid potassium salt | 1:1 | 1:1.30 | chelate |
| Quinic acid ammonium salt | 1:1 | 1:1.54 | chelate |
| Hydroxy citric acid | 1:3 | 1:1.85 | chelate |
| ammonium salt | | | |

Experimental molar ratios were calculated from neutralization to P^{H} = 7.5 of free protonated organic acid (H+) with sodium, potassium or ammonium hydroxides.

Quinic acid itself as the free acid (H+)-or-the hydrolyzed quinic acid esters to release quinic acid, per se, treated with excess ammonia (10% ammonia, for example, for 2 hours, for example) each generates quinic acid ammonia chelate described and characterized herein as efficacious in vivo.

Crude plant extracts or bioactives containing alpha hydroxy acid analog structures other than esters can also be converted into more efficacious formulations by ammonia treatment described herein.

In addition this invention discloses that other plant water extracts that also include bioactive low molecular weight organic acids can also be conveniently converted into more efficacious chelate forms such as: apple, apricot, garcinia, cranberry, quince, citrus fruits, pineapple, prune, sunflowers, whortleberry, blackberry, red currant, black currant, raspberry, babco, feijoa, kiwano, passion fruit, tamarillo, medlar, persimmon or other plant sources; e.g. that are also known to contain hydroxylated or carboxylated organic acids such as but not limited to ascorbic, fumaric, glutaric, lactic, malic, oxalic, tartaric, citric, alpha hydroxy citric, quinic, shikimic, cinnamonic, salicylic, caffeic, hippuric, benzoic, and phenolic acids.

The present invention is also directed to processes to convert substantially all forms of quinic acid in plant material into a quinic acid chelate, particularly quinic acid ammonium chelate, and to the related production of improved medicinal compositions which exhibit increased biological efficacy and decrease toxicity. Particularly preferred compositions of the present invention comprise a substantial amount or at least an effective amount of least one quinic acid chelate to exhibit at least one biological activity property described herein.

Substantial amount, as used herein, refers to compositions wherein a quinic acid chelate represents more than 5% of all forms of quinic acid present in the composition, preferably more than 15%, and most preferably more than 25%.

Preferrably, at least one quinic acid chelate is the majority form of quinic acid that is present in the composition. Majority form, as used herein, refers to compositions wherein a quinic acid chelate represents more than 50% of all forms of quinic acid present in the composition, preferably more than 60%, and most preferably more than 70%.

Compositions are preferred wherein at least one quinic acid chelate is the substantially major form of quinic acid that is present in the composition. Substantially majority form, as used herein, refers to compositions wherein a quinic acid chelate represents more than 50% of all forms of quinic acid present in the composition, preferably more than 60%, and most preferably more than 70%.

Compositions are preferred, for example, wherein quinic acid ammonium chelate is the substantially major form of quinic acid that is present in the composition or wherein quinic acid ammonium chelate is the only form of quinic acid that is substantially present in the composition.

Quinic acid ammonium chelate as the only form that is substantially present, as used herein, refers to compositions wherein a quinic acid chelate represents more than 90% of all forms of quinic acid present in the composition, preferably more than 95%, and most preferably more than 99%.

Compositions are described herein, for example, wherein quinic acid ammonium chelate is present as substantially the only form of quinic acid in the composition.

The present invention is also directed to the isolation, purification and structural identification of the bioactive components of water plant extracts. One class of bioactive components previously identified in Uncaria extracts were identified as CAEs (carboxy alkyl esters) including quinic acid lactone, carbohydrate esters of quinic acid ranging in molecular weight from at least 271 to >10,000, and other related quinic acid esters (e.g. tannins and chlorogenic. The present invention now further identifies the bioactive components of Uncaria to include quinic acid and quinic acid chelates including quinic acid ammonia chelate.

This invention is focused on quinic acid and other natural occurring organic acids such as ascorbic, fumaric, glutaric, lactic, malic, oxalic, tartaric, citric, alpha hydroxy citric, quinic, shikimic, cinnamonic, salicylic, caffeic, hippuric, benzoic, and phenolic acids as bioactive ingredients of numerous plant extracts. Disclosed herein is the fact that it is not organic acid esters or salts that are preferred structures for rendering efficacy to plant extracts but rather their abilities to form chelates.

Because both quinic acid and hydroxycitric acid (garcinia), for example, are more biological effective in their ammoniated chelate forms, then it was reasoned that there may be a manufacturing benefit to formulating quinic acid analogs specifically, and other natural occurring hydroxylated and carboxylated organic acids in plant extracts in general, into their chelated forms by direct treatment of water extracts of cat's claw specifically, and in general other plant extracts, with molecular saturating levels of ammonia.

### Carboxy Alkyl Esters (CAEs) Found in Water Soluble Extracts of Cat's Claw are Quinic Acid Carboydrate Esters

### The Quinic Acid Moiety and Ester Linkage

The CAE present in C-Med-100 (example water soluble Uncaria extract) are quinic acid esters. The presence of the ester linkage to quinic acid is supported by the fact that numerous peaks can be seen in the HPLC chromatograms that are either depleted or disappear following base hydrolysis. This identifies those compounds as esters because of their sensitivity to base hydrolysis. These data confirm that the CAE in Cat's Claw water extracts are in fact QA esters.

### The Alcohol Moiety of the Ester

Attempts to purify the QA esters from C-Med-100, for example, have previously been demonstrated to be extremely difficult, principally due the heterogeneous nature of the esters that could be identified as abundant in C-Med-100, for example. There were at least 5 major base-peaks found in C-Med-100, for example. There are around 10 in Quin+, described herein, that are sensitive to base hydrolysis. Therefore, as further described herein, it is highly unlikely that any one purified QA ester would be able by itself to account for most of the efficacy of Cat's Claw water extracts. The heterogeneity in QA ester structures originate from the alcohol portion of the molecule, and do not contribute in a major way to the efficacy of Cat's Claw products. QA by itself has comparable efficacy to C-Med-100, for example, in vivo.

NMR studies establishing the presence of quinic acid in hydrolysates of Cat's Claw were also useful in identifying the appearance of another major component. This structure's NMR analysis was consistent with it being a disaccharide. The simultaneous appearance of quinic acid and a disaccharide after base hydrolysis of a Cat's Claw extract, was the first indication of the true structure of QA esters in Cat's Claw; namely QA carbohydrate esters. This has been further substantiated in two key ways. First of all glucouronidase is an enzyme well know to attack the glycosidic linkage of carbohydrate polymers breaking them down to smaller sub-units such as monosaccharide, disaccharide and oligosaccharide fragments. The QA esters in Cat's Claw were carbohydrate esters varying under natural condition in chain length and branches are shown by the heterogeneous nature of the QA esters illustrated by glucouronidase treatment. These results are consistent with partially digested molecules produced that are enriched for quinic acid thus moving closer to the retention time of quinic acid (1.96-2.14 min).

Described herein is a process for the production of an isolated medicinal composition for administration to mammals which comprises an effective amount of a free acid, salt or chelate of at least one naturally occurring form of an alpha hydroxyl organic acid comprising: combining at least one naturally occurring form of an alpha hydroxyl organic acid with an amount of strong base (such as 1-10% ammonia, 1-5 M NaOH, 1-5 M KOH, 1-5 M calcium hydroxide in an aqueous solution, wherein a total content of alpha hydroxy organic acids amounts to between about 0.5% and about 35% w/w of the solution within hours, which is adequate to hydrolyze substantially all forms of alpha hydroxyl organic acid esters present to free acids and to convert them to chelates. By acidifying the base-treated solution to a pH of about 7.5 all the salt and chelate forms are substantially converted into only free acid forms, and then the resulting free acid forms are either optionally lyophilized into a dry composition or further converted chelates by neutralization with base.

A total content of alpha hydroxyl organic acids amounts may, for example, be between about 5% and about 35% w/w of the solution.

Practically any laboratory acid known to one of ordinary skill, including, but not limited to hydrochloric acid, sulfuric acid, acetic acid, tartaric acid, lactic acid, propionic acid, citric acid, or nitric acid for example, may be used to “neutralize” the solution. A pH of about 7.5 is preferred.

A process of the present invention is, for example, wherein the base is selected from the group consisting of NaOH, KOH, zinc hydroxide, calcium hydroxide, and NH₄OH and is added to effect a concentration in the aqueous solution within the range of about 0.5M to about 5M for a time between about 15 minutes and about four hours.

The resulting composition may be lyophilized for the production of dosage forms, e.g., for the combination with a pharmaceutically acceptable carrier (e.g., sterile deionized water). A preferred dosage for lyophilized medicinal compositions discussed herein to confer the biological effects discussed herein is between about 0.5 to about 5 mg/kg body weight of human. Preferrably between about 1 mg/kg to about 3 mg/kg body weight. Medicinal compositions described herein may be preferably formulated in water-based drinking beverages, e.g., water, in about 0.5 mg/ml to about 5 mg/ml. Preferrably between about 1 mg/ml to about 3 mg/ml.

A process for the production of an isolated medicinal composition is preferred wherein at least one naturally occurring form of a alpha hydroxyl organic acid is selected from the group consisting of an ester, a carboxy alkyl ester, salt, chelate and a free acid. For example, wherein at least one naturally occurring form of an organic acid is a naturally occurring form of an acid selected from the group consisting of quinic, alpha hydroxyl citric, ascorbic, fumaric, glutaric, lactic, malic, oxalic, tartaric, citric, citric, quinic, shikimic, cinnamonic, salicylic, caffeic, hippuric, benzoic, and phenolic acids.

Processes of the present invention are preferred wherein the naturally occurring form of an alpha hydroxyl organic acid is selected from the group consisting of quinic acid and alpha hydroxy citric, and the base is ammonium hydroxide.

Processes described herein for the production of an isolated medicinal composition for administration are particularly preferred which produce an effective amount of a quinic acid chelate wherein a ratio of quinic acid to ammonium ion is about 1:1.54. Processes of the current invention are preferred which further comprises the step of combining an effective amount of the lyophilized composition with a pharmaceutically-acceptable carrier suitable for oral administration to a mammal.

Acid/Base Conversion of all Forms of Quinic Acid in Uncaria Extracts, for Example, in Situ into More Bioactive Forms (Production of Nutraceutical Composition Quin+)

A chemical principle to be taken advantage of in preparing nutraceutical products isolated from Uncaria, for example, is alteration of the pH. Significant advantage to the efficacy of natural product is achieved by: (1) Optimize the pH at which the bioactive components in the extract are effective, (2) Converting bioactive acids or bases present in nutraceuticals having bioactive forms while reducing toxicity. As disclosed herein Uncaria products, for example, are employed to produce an embodiment of the invention, “Quin+”, for example, ammonium hydroxide treatment to form a chelate. Upon the treatment of QA with ammonia either an ammonium salt or ammonium chelate is formed depending upon the conditions of treatment. If QA is neutralized to pH 7-7.5 a 1:1.54 molar ratio (quinic acid:ammonia ions) of an ammonium chelate is established.

### Development of QA Ammonium Chelates

Quin+ formulations were created as an improvement over the previous Cat's Claw products, including C-Med-100, that quantified CAE and not alkaloids as the primary active ingredients in the Uncaria water extract. The significant improvement of Quin+ over C-Med-100 in the bioavailability of the active ingredient as well as efficacy is documented herein by the following data.
1. The yield of extract from crude bark is about double that found for C-Med-100, being 13.2% instead of 5.2%.
2. Quantitative estimation of CAE or QAE in Quin+ are 5-10 times more abundant than in C-Med-100.
3. Quin+contains QA esters not even present in C-Med-100, e.g., QA esters >10,000 Mw.
4. Quin+comprises a significantly increased amount of the active ingredient rather than a heterogeneous group of QA esters.
5. Once Quin+ has been created its bioactive component QA-H+, is easily converted further into an even more bioactive state by the one-step procedure of converting QA-H+ to QA-NH₄+ by neutralization of Quin+ with ammonium hydroxide

During production, due to the lack of size exclusion, extract described herein, employed in the production of QUIN+ formulations, exhibits 3 times more QA esters that C-Med-100. One of the esters, for example, in the production of QUIN+ exhibits a molecular weight >10,000, accounting for about 15% of the QA esters. The Seliwanoff's reagent used was designed as a colormetric procedure to determine the qualitative nature of carbohydrates irregardless of their size. The analysis of the presence of carbohydrates in the various samples tested was in accordance with both the amount of QA esters present and their size. In summation, various sized carbohydrates were apparently present in every Cat's Claw water extract in relation to the known QA ester content.

### QUINPLUS™ (QUIN+) (Ammonia Treated Plant Extracts)

Described herein are methods to produce nutraceutical compositions from uncaria, for example, which compositions have a significantly increased amount of bioavailable quinic acid, per se.

All forms of quinic acid includes complexed quinic acid, e.g., quinic acid esters (e.g., carbohydrate esters, carboxy alkyl esters (CAEs)), tannins and chlorogenic acid, for example.

The methods described herein, however, may also be similarly applied to other plant materials, garcinia for example, which contain other related organic acids.

In a preferred embodiment of the present invention, these extracts dissolved in water are further treated with approximately 1-10% ammonia to convert the carboxy organic acids that may be present in the extracts as esters, salts or chelated forms into the preferred molecular equilibrated form of ammonia chelates by removal of excess ammonia via freeze drying the extracts. Here it is disclosed that ammonium ions both enhance the nutritive value and the efficacious modes of action of carboxy organic acid bioactive components present in the plant extracts.

Fundamentally, all forms of in situ quinic acid present in plant extracts, preferrably aqueous uncaria extracts as described herein, are converted to (released as) the free (quinc) acid as the active ingredient. Ammonia treated aqueous uncaria extracts are preferred which produce an efficacious ammonium chelate of quinic acid.

In a preferred embodiment of the present invention, these extracts dissolved in water are further treated with 1-10% ammonia to convert the carboxy organic acids that may be present in the extracts as esters, salts or chelated forms into the preferred molecular equilibrated form of ammonia chelates by removal of excess ammonia via freeze drying the extracts.

Water extraction of uncaria material for the production of C-MED-100, for example, is well known in the art. U.S. Pat. Nos. 6,039,949; 6,238,675, and. 6,361,805 are herein incorporated by reference. The present invention is directed to processes to convert substantially all forms of quinic acid in extracted plant material, particularly water extracted uncaria material as known in the art, and as described herein, into free quinice acid or chelate forms, particularly preferred is quinic acid ammonium chelate, and to the related production of improved medicinal compositions which exhibit increased biological efficacy and decrease toxicity wherein quinic acid chelates are the forms of quinic acid that are substantially present. Size exclusion, over 10 kd or 12 kd, for example, after water extraction is not necessary in the production of compositions described herein. Accordingly, large molecular weight forms of complexed quinic acid are available in the extract for hydrolysis and release of the free acid that are not available if the previously practiced size exclusion step is performed.

An example water extraction process for producing a primary extract of water-soluble phytomedicinal compounds comprises combining homogenized (ground or minced) uncaria plant material with water, in a ratio of plant material to water within a range of about 1:5 to about 1:50, at a temperature between about 75° C. and about 100° C. for a period of time to solubilize a substantial portion of thermal aqueous extractable phytocompounds present in the plant material, removing the particulate matter, to produce a composition of water-soluble phytomedicinal compounds. The process is preferred wherein the plant material is selected from the group consisting of leaves, bark, flowers, roots, stems, and fruit. The process is preferred wherein the plant material is selected from the group consisting of bark, roots, and stems. The process is preferred wherein the ratio of plant material to water is within a range of about 1:25 to about 1:35, and the temperature is between about 95° C. and about 100° C., and the period of time is between about 1 hour and about 6 hours.

A process for the production of an isolated medicinal composition which comprises an effective amount of a salt or chelate of a free acid of at least one naturally occurring form of an alpha hydroxyl organic acid is disclosed and claimed herein which comprises combining an aqueous extract of plant material, wherein a total content of alpha hydroxyl organic acids in the extract amounts to between about 0.2% and about 35% w/w of the extract, with an amount of base in an aqueous solution for a time to hydrolyse substantially all forms of alpha hydroxyl organic acids present, neutralizing the solution to a pH between about 6.9 and about 7.6 to yield a free acid, salt or chelate of free acids of substantially all naturally occurring forms of alpha hydroxyl organic acids, and optionally lyophilizing the solution to produce an isolated medicinal composition.

Processes are preferred wherein the plant material is selected from the group consisting of uncaria, garcinia, cranberry, and coffee and the base is selected from the group consisting of NaOH, KOH, and NH₄OH and is added to effect a concentration in the aqueous solution within the range of about 0.5 M to about 5 M for a time between about 15 minutes and about four hours. Processes are particularly preferred wherein the plant material is uncaria (cat's claw) and wherein the isolated medicinal composition comprises an effective amount of a quinic acid chelate wherein a ratio of quinic acid to ammonium ion is about 1:1.54.

Processes further comprises the step of combining an effective amount of the lyophilized composition with a pharmaceutically-acceptable carrier to produce a formulation suitable for oral or systemic administration to a mammal.

If plant materials such as larch, pine bark, red wine, garcinia, green tea, bilberry, black cohosh, cayene, chamomile, chaste tree, cranberry, echinacea, eleuthero, ephedra, evening primrose, feverfew, flax, garlic, ginger, ginkgo, ginseng, golenseal, hawthorn, horse chestnut, kava, licorice, milk thistle, peppermint, saw palmetto, saint john's wort, black tea, valerian apple, apricot, quince, citrus fruits, pineapple, prune, sunflowers, whortleberry, blackberry, red currant, black currant, raspberry, babco, feijoa, kiwano, passion fruit, tamarillo, medlar, or persimmon shown to contain hydroxylated or carboxylated organic acids are hot water extracted, which has been common historical practice for medicinal use, phytomedicinal preparations are prepared of the aforementioned plant extracts having potent immuno, anti-tumor, anti-inflammatory, and DNA repair enhancing properties. In a preferred embodiment of the present invention, these extracts dissolved in water are further treated with 1-10% ammonia to convert the carboxy organic acids that may be present in the extracts as esters, salts or chelated forms into the preferred molecular equilibrated form of ammonia chelates by removal of excess ammonia via freeze drying the extracts. Here it is disclosed that ammonium ions alone both enhance the nutritive value and the efficacious modes of action of carboxy organic acid bioactive components present in the plant extracts such as inhibiting NF-kB or inducing growth arrest.

The uncaria water extract (C-MED-100, for example) is subject to a hydrolysis step. The extract is subject to about IN strong base, for example, for about two hours, for example, to release the free acid (quinic acid). At least one base is used. Ammonium hydroxide is preferred. Other bases such as sodium hydroxide or potassium hydroxide may also be employed. This converts the extract to about pH 11-12 and hydrolyses all esters of quinic acid to QA+ carbohydrate.

Uncaria water extracts are treated in situ with strong base (1 M NaOH, for example) for about 2 hours, for example, then neutralized to about pH 7-7.5 with HCl, for example. Substantially all QA esters are converted to free quinic acid-H+, and then likewise stepwise converted into any desired salt form or chelate by neutralization with the desired base (e.g. ammonium hydroxide, NaOH, KOH, etc). Once converted into QA-H+ (free acid of quinic acid) all the original sources of quinic acid (all forms of quinic acid) “QA content” in the extract is then placed in an elevated efficacious state by conversion preferably to QA-NH₄+ chelate. Moreover, NH₄Cl by itself is a powerful antioxidant which in turn supports the advantage of forming QA-NH₄+. A bioactive composition of Cat's Claw (uncaria) is standardized herein from both a chemical composition and pharmacological/efficacy points of view.

### Example Pharmacological Value

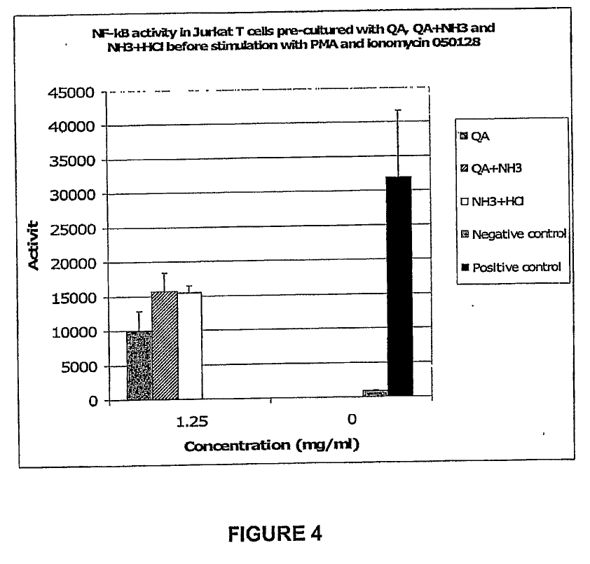

Previous reports in the scientific literature have established that both Cat's Claw extracts and quinic acid inhibit NF-kB in a dose dependent manner. Aquilar, J L et al. J. Ethanopharmacology 81: 271-276, 2002; Tak, P P et al. J Clin Invest 107: 7-11, 2001; Akesson et al. Int Immunopharmacol 3: 1889-1900, 2003; Akesson et al. Int Immunopharmacology 5: 219-22, 2005. These data do not include the concept that certain natural occurring organic acids such as quinic acid, as well as other natural occurring simple alpha hydroxy acids are, can also be converted into salts and chelates with ammonia, which in turn are highly effective at inhibiting NF-kB. Ammonium ions have been shown to be effective inhibitors of lysosomal generation of oxidative stress, and as such are synergistic to inhibition of NF-kB. Ogawa, Y et al. Int J Mol Med 14(6): 1007-1013, 2004. Thus ammonium chelates and salts are more effective inhibitors of NF-kB because ammonium ions independently of quinic acid or other alpha hydroxyl acids capable of delivering NF-kB inhibition to cells via the salt or chelated formulations of organic acids such as quinic acid. The data disclosing this principle of the invention are presented in FIG. 4.

In another embodiment, the present invention comprises a pharmaceutical composition comprising a pharmaceutically effective amount of the bioactive hydroxylated and carboxylated organic acid chelates originally identified in plant extracts as organic acids and a nontoxic inert carrier or diluent. The present invention also includes embodiments which comprise using the pharmaceutical composition to (i) enhance the immune competency of a mammal by inhibiting TNF-α production or inducing apoptosis of white blood cells, comprising administering the pharmaceutical composition in an amount effective to inhibit TNF-a production or to induce apoptosis of white blood cells; (ii) treat disorders associated with the immune system of a mammal by inhibiting TNF-α production or inducing apoptosis of white blood cells, comprising administering the pharmaceutical composition in an amount effective to inhibit TNF-a production or to induce apoptosis of white blood cells; (iii) inhibit the inflammatory response of a mammal by inhibiting TNF-α production or inducing apoptosis of white blood cells, comprising administering the pharmaceutical composition in an amount effective to inhibit TNF-α production or to induce apoptosis of white blood cells; (iv) treat disorders associated with the inflammatory response of a mammal by inhibiting TNF-α production or inducing apoptosis of white blood cells or increasing white blood cells (WBC) in vivo after chemotherapy-induced leucopenia, comprising administering the pharmaceutical composition in an amount effective to inhibit TNF-α production or to induce apoptosis of white blood cells; (v) enhance the anti-tumor response of a mammal by inducing apoptosis of tumor cells, comprising administering the pharmaceutical composition in an amount effective to induce apoptosis of tumor cells; (vi) treat disorders associated with the response of a mammal to tumor formation and growth by inducing apoptosis of tumor cells, comprising administering the pharmaceutical composition in an amount effective to induce apoptosis of tumor cells; and (vii) enhance the DNA repair processes of a mammal, and, thus, provide anti-mutagenic activity important to treating aging disorders.

Independent inhibition of NF-kB by either QA or NH₄+ ions. Human Jurkat T cells were incubated together with either QA, QA-NH₄+ or NH₄Cl, and the degree of inhibition of NF-kB evaluated as described elsewhere. See, FIG. 4. Parra E, et al., 1997. Mol Cell Biol 17:1324-23; Akesson, C., et al., Int. Immunopharmacol., 5(1):219 (2005).

Products produced by processes of the present invention are important subject matter of the invention disclosed and claimed herein.

### QUINMAX™

Ammonium chelate of quinic acid is indeed the preferred composition of quinic acid experimentally determined as a 1:1.54 molar ammonia chelate when formed by treatment with saturated amounts of 1-10% ammonia to convert quinic acid to pH=7.5.

The discovery began in trying to explain the biological activity of a hot water extract of Cat's Claw bark, Uncaria tomentosa, called C-Med-100. This extract has been demonstrated to be efficacious at stimulating DNA repair. After this initial biological characterization, the applicant has set forth to identify the natural product behind DNA repair enhancement as a potential anti-aging therapy. A series of chemical studies have led scientists to first identify carboxy alkyl esters (CAEs) as the bioactives and then it was followed by quinic acid esters (QAEs) being identified as the only bioactive CAEs in cat's claw water extracts. Cat's Claw QAEs were bioactive both in vitro and in vivo. However when acid or base hydrolyzed into quinic acid, and without having the alcohol moiety of the QAEs present, quinic acid by itself had as much DNA repair enhancing activity in vivo as did the QAEs. Hence the conclusion that quinic acid was the final biological active form of Cat's Claw. However, quinic acid is present in water extracts of Cat's Claw as QAES. QAEs are in effect in a pro-metabolite form since the QAEs would be hydrolyzed to quinic acid in the gastrointestinal tract.

Chronological discoveries are presented below:
1. 1967—The perceived active ingredients of Cat's Claw were the oxindole alkaloids first presented by Dr Klaus Keplinger. Keplinger, K, et al., 1999. Uncaria tomentosa (Wild) DC-ethno medicinal use and new pharmacological, toxicological and botanical results. J Ethanopharmacology 64: 23-34.
2. 2000-2000—Cat's Claw extracts such as C-Med-100 were essentially devoid of alkaloids, and yet were highly efficacious thus eliminating alkaloids as the primary active ingredients. Sheng, Y, Pero, R W, Wagner, H. 2000. Treatment of chemotherapy induced leucopenia in the rat model with aqueous extract from Uncaria tomentosa. Phytomedicine 7:137-143; Sandoval, M, et al., 2002. Antiinflammatory and antioxidant activities of Cat's Claw (Uncaria tomentosa and Uncaria guianensis) are independent of their alkaloid content. Phytomedicine 9: 325-337.
3. 1998-2003—Water extracts of Cat's Claw prevented or controlled ulcerative colitis (inflammatory responses), osteoarthritis/joint pain, tumor cell growth, weight gain, ozone injury, DNA damage/cell death, chemotherapeutic-induced leucopenia and dementia/Alzheimers. Sandoval-Chacon, M, et al., 1998. Anti-inflammatory actions of cat's claw: the role of NF-kappa B. Alimentary Pharmacological Therapy 12: 1279-1289; Piscoya, J, et al., 2001. Efficacy and safety of freeze dried cat's claw in osteoarthritis of the knee: mechanisms of action of the species Uncaria guianensis. Inflammation Res 50: 442-448; Sheng, Y., Pero, R. W., et al., 1998. Induction of apoptosis and inhibition of proliferation and clonogenic growth of human leukemic cell lines treated with aqueous extracts of Uncaria Tomentosa, Anticancer Research 18:3363-3368; Pero, R W, et al., Formulation and clinical evaluation of combining DNA repair and immune enhancing nutritional supplements. Phytomedicine 12(4): 255, 2005; Sheng, Y, Pero R W, et al., 2000, Treatment of chemotherapy-induced leukopenia in the rat model with aqueous extract from Uncaria Tomentosa. Phytomedicine 7(2): 137-143; Cisneros, F J, et al., 2005, An Uncaria tomentosa (Cat's Claw) extract protects mice against ozone-induced lung inflammation. Journal of Ethanopharmacology 96: 355-364; Castillo and Snow U.S. Pat. No. 6,346,280 issued February 2002) whereas DNA repair and immune cell function were enhanced (Lamm, S., Sheng, Y., Pero, R. W. 2001. Persistent response to pneumococcal vaccine in individuals supplemented with a novel water soluble extract of Uncaria tomentosa, C-Med-100. Phytomedicine 8(4): 267-274; Sheng, Y., Bryngelsson, C., Pero, R. W. 2000. Enhanced DNA repair, immune function and reduced toxicity of C-MED-100™, a novel aqueous extract from Uncaria tomentosa. Journal of Ethnopharmacology 69:115-126; Sheng, Y., Li, L., Holmgren, K., Pero, R. W. 2001. DNA repair enhancement of aqueous extracts of Uncaria Tomentosa in a human volunteer study. Phytomedicine 8(4): 275-282; Åkesson, C, Lindgren, H, Pero, R W, Leanderson, T, Ivars, F. 2003. An extract of Uncaria tomentosa inhibiting cell division and NF-kappa B activity without inducing cell death. Int Immunopharmacol 3: 1889-1900).
4. 2000-2002—CAEs (carboxy alkyl esters) as a general chemical class of natural compounds were shown to be responsible for Cat's Claw efficacy. Sheng, Y, Pero R W, et al., 2000. Treatment of chemotherapy-induced leukopenia in the rat model with aqueous extract from Uncaria Tomentosa. Phytomedicine 7(2): 137-143.
5. 2005—Quinic acid esters were shown to be the main type of CAE responsible for the efficacy of Cat's Claw. Sheng, Y, et al., R W. 2005. An active ingredient of Cat's Claw water extracts. Identification and efficacy of quinic acid. Journal of Ethanopharmacology 96(3): 577-584.
6. 2005—Quinic acid occurs ubiquitously in food and natural supplements as esters, salts or the free acid principally because it is the key biosynthetic intermediate to aromatic (i.e. conjugated double bond ring system) compound production in plants (Herrmann, K. M., Weaver, L. M. 1999. The shikimate pathway. Annual Review of Plant Physiology and Plant Molecular Biology 50: 473-503). Hence, because of the strong pH=1 in the stomach that would breakdown esters, and because of the occurrence of high levels of non-specific esterases in humans that would in turn also metabolize esters to quinic acid, then it followed that natural occurring quinic acid esters were likely prodrugs being converted to the ultimate bioactive form of quinic acid by the body's metabolism.

Quinic acid cannot be produced by warm blooded animals. It is not synthesized in the body, but it is present in small amounts in the diet, and can protect the DNA health of individuals against major disease.

### Ultrapurified, Efficacious Natural Product

A process is particularly preferred for the production of an isolated medicinal composition which comprises an effective amount of a quinic acid chelate comprising: combining substantially pure quinic acid, with ammonium hydroxide in an aqueous solution sufficient to reach a pH between about 6.9 to about 7.6, to yield an ammonium chelate of quinic acid wherein a ratio of quinic acid to ammonium ion is about 1:1.54. Processes are preferred wherein a solution of ammonium hydroxide, between about 1% and about 10% in concentration, is added to an aqueous solution of quinic acid which comprises between about 5 g to about 30 g quinic acid per 100 ml, in a sufficient amount for the solution to reach a pH between about 7.4 and about 7.6 within a time period between about 15 minutes to about four hours.

QUINMAX™ is fundamentally substantially pure quinic acid ammonia-treated to form a substantially pure ammonium chelate in about a 1:1.6 (actually 1:1.54) molar ratio at a physiological pH. Disclosed and claimed herein are processes for the production of an isolated and purified composition of an efficacious ammonium chelate of quinic acid. Compositions described herein are produced, for example, by converting substantially pure D-Quinic acid to the ammonium chelate in about a 1:1.6 molar ratio in an aqueous medium using ammonium hydroxide within the range of about pH 7 to about pH 7.5. A pH of about 7.5 is preferred. However, quinic acid ammonium chelates described herein may be produced, for example, at pH 6.9, 7, 7.1, 7.2, 7.3, 7.4, 7.5, and 7.6, and at all pH values in between. Disclosed herein are isolated pharmaceutical or nutraceutical compositions which comprise a significant and effective amount of the ammonium chelate of quinic acid. Quinmax™ is the 1 (QA):1.6(NH₄+) molar ratio of the ammonium chelate of quinic acid.

Quinmax™, the 1:1.54 molar ratio of the ammonium chelate of quinic acid, to be as efficacious as Cat's Claw water extracts such as C-Med-100 at enhancing DNA repair, inhibiting DNA damage and preventing cell death.

Quinmax™ is substantially pure quinic acid neutralized with aqueous ammonia to pH=7.5. As a result, in water ammonium ions are also generated which in themselves have potent biological functions. Ammonium ions, for example, stimulate protein synthesis in the GI tract, neutralize lysosomal vesicles thus preventing oxidative stress DNA damage, and inhibit NF-kB mediated inflammatory responses. These are examples of additional biological properties of Quinmax™, which is devoid of any added-on toxicity. Quinic acid, for example, at 2700 μg/ml did not demonstrate any toxicity when evaluated by mutagenicity in the Ames Assay. Jacobsen, L B, Richardson, C L, Floss, H G. 1978. Shikimic acid and quinic acid are not mutagenic in the Ames assay. Lloydia 41(5): 450-452. The safety is presented in Table 2. Here the most important consideration is to compare the highest doses of quinic acid exposure to those shown to have efficacy in various animal models. It is quite apparent that quinic acid doses that far exceed the known efficacious doses of quinic acid have been used in the various animal model systems. Hence the margin of safety for in vivo treatment with quinic acid has been consistently shown for doses that are predicted to be efficacious in humans.

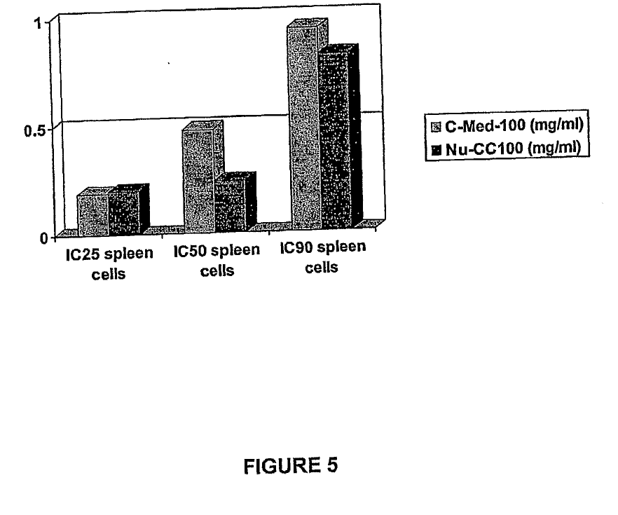

The excess ammonia was removed by freeze drying. The comparison of C-Med-100 with C-Med-100 ammonia chelate is presented in FIG. 5 as IC values.

The known toxicity or safety of quinic acid is presented in Table 2. Here the most important consideration is to compare the highest doses of quinic acid exposure to those shown to have efficacy in various animal models. It is quite apparent that quinic acid doses that far exceed the known efficacious doses of quinic acid have been used in the various animal model systems. Hence the margin of safety for in vivo treatment with quinic acid has been consistently shown for doses that are predicted to be efficacious in humans. Moreover, Quinmax™ is quinic acid neutralized with aqueous ammonia to pH=7.5. As a result, in water ammonium ions are also generated which in themselves have potent biological functions; e.g. stimulates protein synthesis in GI tract, neutralizes lysosomal vesicles preventing oxidative stress DNA damage, and itself inhibits NF-kB mediated anti-inflammatory responses. Fuller, M F, Reeds, P J. 1998. Nitrogen cycling in the gut. Ann Rev Nutr 18: 385-411; Seglen, P O. 1983. (59) Inhibitors of lysosomal function. Methods of Enzymology 96: 737-764. These were additional biological properties added to Quinmax, but were devoid of any added-on toxicity.

TABLE 2
Safety, toxicity, and efficacy of Quinic acid and Quinmax ™

| | | | | |
|---|---|---|---|---|
| | Highest | | | |
| Species | Dose Tested | Toxicity | Efficacy | Reference |

Mouse:

| | | | | | |
|---|---|---|---|---|---|
| Quinic acid | 500 | mg/kg | None | 250 mg/kg | Akesson et al 2005 |
| (drink water) | | | | | |

Rat:

| | | | | | |
|---|---|---|---|---|---|
| Quinmax ™ | 200 | mg/kg | None | ≦200 mg/kg | Sheng et al 2005 |
| QA-NH₄+ chelate, | | | | | |
| 1:1.6 molar ratio | | | | | |
| (oral) | | | | | |
| Quinic acid | 200 | mg/kg | None | ≦200 mg/kg | Sheng et al 2005 |
| (oral) | | | | | |
| Quinic acid | 320 | mg/kg | None | n.d. | Gonthier et al 2003 |
| (oral) | | | | | |
| Quinic acid | 400 | mg/kg | None | n.d. | Indahl et al 1973 |
| (oral) | | | | | |
| Quinic acid | 2900 | mg/kg | None | n.d. | Seifter et al 1971 |
| (oral) | | | | | |

Guinea Pig:

| | | | | | |
|---|---|---|---|---|---|
| Quinic acid | 1000 | mg/kg | None | n.d. | Bernhard et al 1955 |
| (oral) | | | | | |

Lower animals (rabbits, hamsters, guinea pigs, lemmings, pigeons,
dogs, cats, ferrets, hedgehogs, fruit bats, rats and mice:

| | | | | | |
|---|---|---|---|---|---|
| Quinic acid | 300-600 | mg/kg | None | n.d. | Adamson et al 1970 |
| (oral) | | | | | |

Human:

| | | | | | |
|---|---|---|---|---|---|
| Quinmax | 70 | mg/kg | None | n.d. | University of Lund |
| (QA-NH4+ chelate, | | | | | human volunteer |
| 1:1.6 molar ratio) | | | | | n = 2 |
| (oral) | | | | | |
| Quinic acid | 90 | mg/kg | None | n.d. | Beer et al 1951 |
| (oral) | | | | | Adamson et al 1970 |

Primates (rhesus, green, spider, squirrel monkeys):

| | | | | | |
|---|---|---|---|---|---|
| Quinic acid | 350 | mg/kg | None | n.d. | Adamson et al |
| (oral) | | | | | |
| 1970 | | | | | |

n.d. = not determined

Additional oral administration of high dose QA was carried out after ingestion of 6 gm Quinmax™ in human volunteers at the University of Lund. The purpose of this study was to determine if significant metabolism of QA to hippuric acid occurred when QA was administered in the ammonia chelated form as Quinmax™, a 1:1.6 molar ratio of QA to ammonia ions.

A 65 year old apparently healthy volunteer drank 6 gm Quinmax™ dissolved in 300 ml water over a 15 min period, and then about 40 ml of peripheral blood (4-red topped vacutainers) were allowed to clot at room temperature to prepare serum samples by centrifugation. The serum sampling points were 0.7 hr, 1.7 hr, 2.7 hr, 3.7 hr, 10.5 hr 12.5 hr, 22 hr, 28 hr and 44 hr. 30 ml serum samples were precipitated with 50% ethanol, taken to dryness under a stream of air, and redissolved in 1 ml of methanol for simultaneous HPLC analysis of quinic acid and hippuric acid.

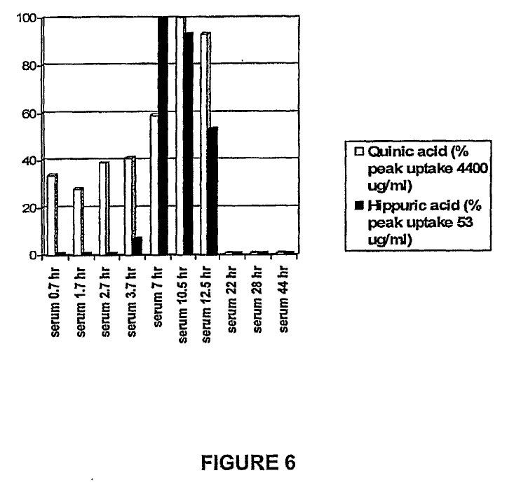

The data are presented in FIG. 6.

The objectives of this experiment were 3-fold. First to determine when peak serum concentrations of QA occurred and how long they would remain detectable. Second was to find out if significant amounts of QA was converted to hippuric acid, Thirdly if Quinmax™, the ammonium chelate of QA and a more efficacious version of QA, could be used in vivo without altering the pharmacokinetics of QA observed in earlier experiments.

The data reported in FIG. 6 establishes peak serum concentrations occurred around 10.5 hr which was reasonably consistent with earlier data, hippuric acid could be measured between 3.7 and 12.5 hr which was synchronized with QA peak serum levels, and the pharmacokinetics of Quinmax™ was similar to QA.

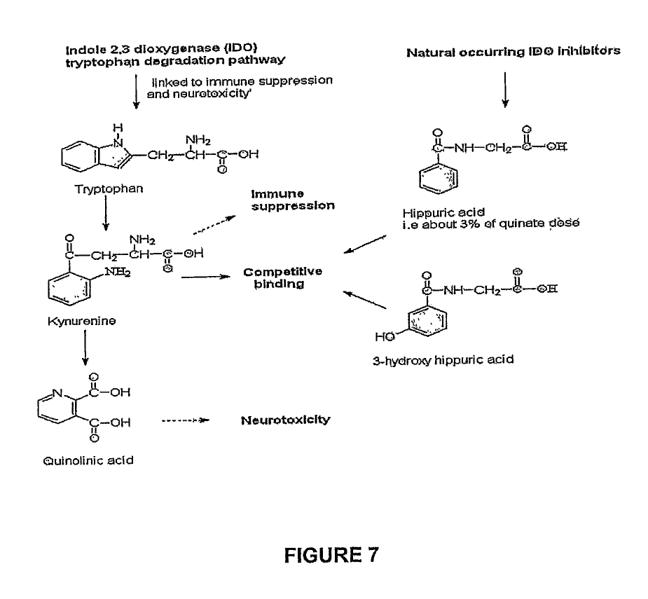

Another important reason for showing that QA could serve as a metabolic source of hippuric acid is shown in FIG. 7.

Quinmax™ can be supplemented in drinking water at a concentration of 2 mg/ml as 500 ml×2 oral doses, for example. Because quinic acid has been dosed in humans up to 6000 mg/day, the supplemented water is safe, colorless and tasteless offered as the quinic acid ammonium chelate.

Quinmax™ is designed as a natural DNA protector. Quinic acid is Vitamin DNA. It mediates its health benefit by enhancing the natural enzymatic process of removing lesions in the DNA structure itself after oral supplementation. Scientists have repeatedly been able to demonstrate that an animal's longevity is predicted by its ability to carry out DNA repair. For example, rodents live only a couple of years compared to humans who can easily live to 90 years, and humans have 16-times the DNA repair capacity that rodents do. Moreover, there is a linear relationship between mammal longevity in general and DNA repair capacity. Clearly mammals have evolved into life as we now know it at least in part by protecting the DNA from damage.

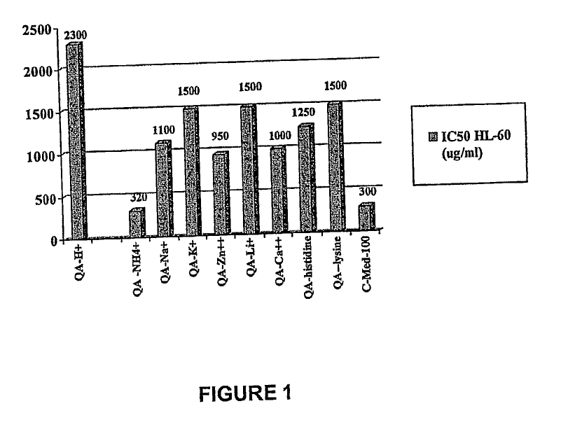

Quinic acid, Quinmax™ and C-Med-100 were all shown to prevent cell death after exposure to a DNA damaging agent, DXR, presumably by removing or otherwise preventing DNA damage (FIG. 1). Rat white blood cells (WBC) or mouse spleen WBC were increased in vivo after quinic acid oral administration either by gavage or in drinking water from 7.2 to >8.2×10⁶ cells/ml or 6.2 to 8.3×10⁹ cells/ml, respectively. These data calculate into a 13-25% increase in the protection of the DNA from becoming damaged and killing the cells. If extrapolated into a lifespan which is logically reasonable, then living to an average age of 90 years would be increased to 112 years by ingesting optimal amounts of quinic acid during one's lifetime.

Immune function enhancement. Because C-Med-100 induced growth responses of immune competent cells are mimicked by quinic acid, then it was observed that quinic acid also induced immune function by increasing the number of fully functional lymphocytes without suppressing antigenic responses to growth stimuli.

Cat's Claw water extracts such as C-Med-100 prevented ozone injury in the lungs of mice, and induced dissolution of amyloid bodies in Alzheimer models (Castillo and Snow U.S. Pat. No. 6,346,280 issued February 2002). Because both quinic acid and C-Med-100 are potent inhibitors of NF-kB then they are also powerful antioxidants, because they in turn stop the production of oxygen radicals via pro-inflammatory cytokine inhibition. Sandoval-Chacon, M, et al., 1998. Anti-inflammatory actions of cat's claw: the role of NF-kappa B. Alimentary Pharmacological Therapy 12: 1279-1289. Hence, quinic acid in any of its natural occurring forms of free acids, esters salts or chelates is expected to be a potent anti-inflammatory.

Anti-tumor activity. As already pointed out the most common form of quinic acid encountered in nature exists as esters, but esters are likely to be hydrolyzed in the stomach and GI tract to quinic acid. Both quinic acid and quinic acid esters have been shown to be effective at controlling tumor growth both by inhibiting proliferation and invasiveness of tumor cell growth. Yagasaki, K, et al., 2000. Inhibitory effects of chlorogenic acid and its related compounds on the invasion of hepatoma cells in culture. Cytotechnology 33(1-3): 229-235; Hata, G, et al., 1992. Synthesis, structure and antitumor activity of a water-soluble platinum complex, 1R,3R,4R,5R-quinato(1R,2R-cyclohexanediamme)platinum (II). Chem Phram Bull (Tokyo) 40(6): 1604-1605.

Moreover, the data strongly support a general health benefit of quinic acid commonly found in nutritious foods. For example, if the average dry weight consumption of food were about 500 mg/day, and if all the food stuffs consumed had a quinic acid content of 20%, then from natural food sources about 100 mg of quinic acid could possibly be ingested. Taking into account that a daily human dose of about 1400 mg would be about optimal, then it follows that the human population would benefit greatly by supplementing Quinmax™ to food or drink.

A preferred dosage for lyophilized medicinal compositions discussed herein to confer the biological effects discussed herein is between about 0.5 to about 5 mg/kg body weight of humans. Preferrably between about 1 mg/kg to about 3 mg/kg body weight. Medicinal compositions described herein may be preferably formulated in water-based drinking beverages, e.g., water, in about 0.5 mg/ml to about 5 mg/ml. Preferrably between about 1 mg/ml to about 3 mg/ml.

### Functional Foods

### Disclosed Herein is a Process to Hydrolyze the Natural Occurring QAEs in Foods to Free Quinic Acid for Reducing Toxicity and Increasing Health Effects

Phenolics Such as hydrolyable tannins (taragallotannins and caffetannins) and Chlorogenic analogs are not only regarded as toxic but they also contain high concentrations of quinic acid in ester linkage with caffeic acid or glucose (e.g. about 50% of chlorogenic acid is quinic acid). Strong base or acid hydrolysis with 1 M NaOH or 1 M HCl, for example, of certain foods generates free quinic acid in that food.

Process of the present invention are disclosed for the production of a functional food which comprises an effective amount of a free acid, salt or chelate of at least one naturally occurring form of an alpha hydroxyl organic acid comprising combining a food, which comprises an amount of alpha hydroxyl organic acids in the food between about 0.2% and about 35% w/w, with a base in an aqueous solution for a time to hydrolyse substantially all forms of alpha hydroxy organic acids in the food, neutralizing the solution to a pH between about 6.9 and about 7.6 to yield a free acid, salt or chelate of free acids of substantially all naturally occurring forms of alpha hydroxyl organic acids in the food, and optionally lyophilizing the solution to produce an isolated medicinal composition.

Processes are preferred, for example wherein the food is selected from the group consisting of apple, apricot, garcinia, cranberry, quince, citrus fruits, pineapple, prune, sunflowers, whortleberry, blackberry, red currant, black currant, raspberry, babco, feijoa, kiwano, passion fruit, tamarillo, medlar, persimmon and coffee and the base is selected from the group consisting of NaOH, KOH, and NH4OH.

### Advantage of Identifying Efficacious Levels of QA in Food and the Formation of Novel Food Additives and Supplements Containing QA Ammonium Chelates

Functional foods are those that encompass potential healthful components including any modified food or ingredient that may provide a health benefit beyond the nutrients as defined by traditional medicinal practice. Healing power of foods is the popular concept of functional foods. As pointed out here and throughout the literature quinic acid esters are major sources of quinic acid occurring naturally in ester linkage mainly with tannins, or chlorogenic acid analogs containing phenols or linked to carbohytraes. Many different forms of quinic acid are a natural component of many foods. If the “released” QA, per se, were present in high enough concentration in edible materials derived from plants, it would convey the property of being a functional food. Many foods have both QAEs, for example, and quinic acid in their compositions. Data presented here illustrates that if the quinic acid content of plants or food is above 0.5% w/w, the corresponding food fundamentally functions as an anti-aging nutraceutical upon human consumption by enhancing DNA repair and immune responsiveness. Plant sources having this minimum concentration or greater would be effective treatments to increase health benefits from stimulating these natural protective processes of the body. Greater than 0.5% quinic acid food content per serving per day would equal to a daily human dose of 1 gm quinic acid per day per 200 gm serving (i.e. calculated from effective rodent doses of 200 mg/kg/day).

TABLE 3
Examples of foods qualifying as functional foods for DNA repair and immune
enhancement based on their quinic acid content being >0.5% per daily
serving (≦150 grams) which has been shown to be efficacious.

| Plant | Plant | % Quinic acid | |
|---|---|---|---|
| Common Name | Scientific Name | (gm/100 gm or ml) | Reference |
| 1. Prunus domestica L | Prunes | 0.7% | van Gorsel et al 1992 |
| 2. Actinidia chinensis Planch | Kiwi | 0.8% | van Gorsel et al 1992 |
| 3. Hippoohae rhamnoides | Sea buckthorn | 2.3% | Beveridge et al 1999 |
| 4. Camellia sinenis | Black/Green tea | 2.0% | Graham et al 1992 |
| 5. Coffea arabica | Coffee | 4.7-5.9% | Engelhardt et al 1985 |
| 6. Vaccinium macrocarpon | Cranberry | 2.7-3.6% | Jensen et al 2002 |
| 7. Vaccinium vitis-idaea | Lingonberries | 2.3-3.2% | Jensen et al 2002 |
| 8. Vaccinium myrtillus | Blueberries | 0.4-0.8% | Jensen et al 2002 |
| 9. — | Wortleberry | 0.5-1.2% | Romero Rodrigues |
| | | | et al 1992 |
| 10. Cyphomandra species | Red/yellow | 0.4-0.8% | Romero Rodrigues |
| | Tamarillo | | et al 1992 |
| 11. — | Sultana | 0.8% | Lewis et al 1995 |

### Food Health Benefits

There are two primary natural sources of quinic acid in foods: (i) Foods having significant quantities of quinic acid stored in ester form as just discussed, and (ii) Foods having efficacious levels of free quinic acid already natural occurring as described. Both food sources occur simultaneously in most plants and are additive to each other. For example, a cup of coffee contains about 13 gms of solids of which about 765 mg are cholorgenic acid analogs and about 50% (388 mg) of that is quinic acid or 0.338 gm/13 gm=2.6% of coffee. Coffee also contains 4.7-5.9% free (non-esterified) quinic acid. Therefore, by hydrolyzing food sources, or by hydrolyzing food sources that have no free quinic but high concentrations of tannins or chlorogenic acid or both, then those foods could be converted into functional foods that have the efficacious health benefit attributed to a quinic acid-mediated response. Examples of food sources that would directly qualify as either a functional food or a food additive by QA-ester hydrolysis increasing free quinic acid in food to achieve an efficacious dose in the gastrointestinal tract. When the quinic acid content of food rises above 0.5% of that absorbed into the gastrointestinal tract then DNA repair and immune enhancement occurs because quinic acid in blood is sufficiently increased. Viewed in this manner quinic acid-containing functional foods are those having >0.5% quinic acid such as prune, kiwi, sea buckthorn, coffee, cranberry, lingonberry, blueberry, wortleberry, red/yellow tamarillo, and sultana. Those having quinic acid content <0.5% are good candidates to become converted to food additives because the quinic acid content could likely be raised to >0.05%. Examples of food additive sources in this category were quince, sunflower, nectarine, peach, pear, plum, honey, black currant, medlar, apricot, asparagus, mushroom and green olive.

### EXAMPLES

### Example I

The inventor has previously disclosed that quinic acid and ammonia-treated quinic acid, and in relation to it's natural cat's claw source (e.g. C-Med-100), can induce growth arrest without cell death and inhibit NF-kB as mechanisms contributing to their use in the treatment of inflammation, immune and DNA repair inhibition, cancer growth and aging (Sheng, Y, Akesson, C, Holmgren, K, Brynegelsson, C, Giampapa, V, Pero, R W. An active ingredient of Car's Claw water extracts. Identication and efficacy of quinic acid. Journal of Ethanopharmacology 92: 577-584, 2005; Akesson, C, Lindgren, H, Pero, R W, Leanderson, T, Ivars, F. Quinic acid is a biologically active component of the Uncaria tomentosa extract C-Med 100%. International Immunopharmacology 5: 219-229, 2005). The active ingredient of water extracts of cat's claw such as C-Med-100 is an analog of quinic acid. The natural form of quinic acid in C-Med-100 is a quinic acid ester. However, quinic acid itself as the free acid (H+) or the hydrolyzed quinic acid ester treated with 1-10% ammonia generates quinic acid ammonia salt or chelate (neither previously contemplated or described), both of which are described and characterized herein as efficacious in vivo. Embodiments of quinic acid esters and/or quinic acid salts of the present invention are also in a chelated complex with cations. The final quinic acid bioactive structure, heretofore, however, has remained undetermined. Here we present data that quinic acid can exist in both salt and chelated structural complexes, and that ammonia-treated quinic acid is indeed the preferred composition of quinic acid experimentally determined as a 1:1.54 molar ammonia chelate when formed by treatment with saturated amounts of 1% ammonia to convert quinic acid to pH=7.5 (Table 4). Furthermore it is taught that other naturally occurring salts such as alpha hydroxyl citric acid present in Garcinia extracts (i.e. Citrimix) also exist in a chelated complex when neutralized with 1% ammonia. In conclusion these data disclose (i) that chelated complexes of simple hydroxylated and carboxylated organic acids often form chelates with a variety of cations but not all of them for example NaOH treatment of quinic acid yields a 1:1 molar salt, and (ii) the ammonium chelate of organic acids is a preferred composition because of it's enhanced nutritive and efficacious value.

TABLE 4
Determination of salts and chelates of some naturally occurring
polyhydroxylated and polycarboxylated organic acids.

| | Molar ratio of | Experimental molar ratio | Determination of |
|---|---|---|---|
| | salt theoretical | of molecular equilibrium | structure existing |
| Organic acid salt | carboxy(−)/cation(+) | in water | in water |
| Quinic acid sodium salt | 1:1 | 1:1 | salt |
| Quinic acid potassium salt | 1:1 | 1:1.30 | chelate |
| Quinic acid ammonium salt | 1:1 | 1:1.54 | chelate |
| Hydroxy citric acid | 1:3 | 1:1.85 | chelate |
| ammonium salt | | | |

Experimental molar ratios were calculated from neutralization to P^{H} = 7.5 of free protonated organic acid (H+) with sodium, potassium or ammonium hydroxides.

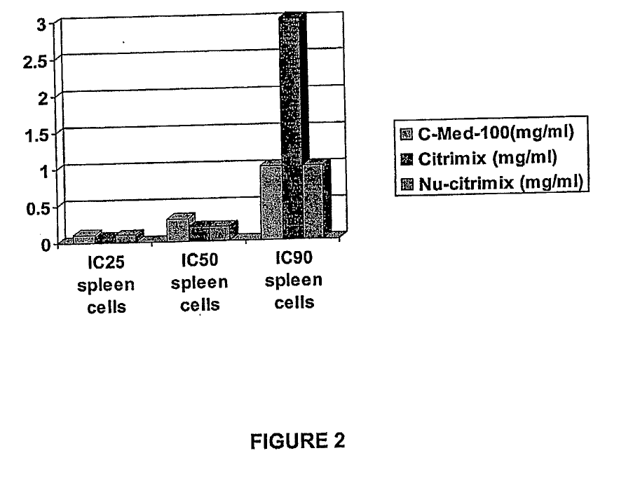

The data presented below in FIGS. 1-2 demonstrate the advantage of ammonia chelates of organic acids in comparison to other cation chelates or salts.

For the study involving quinic acid salts and chelates (FIG. 1) quinic acid was purchased from Sigma (>99%). QA salts/chelates were synthesized by neutralization to pH=7.5 with the appropriate base, i.e., NH₄OH, NaOH, Ca(OH)₂, Zn(OH)₂, LiOH, KOH, lysine or histidine. Serial dilutions of test compounds were added to human HL-60 leukemic cells (0.05×10⁶ cells/ml) in 96-well, flat bottomed microtiter plates to give final concentrations in the cultures up to 3000 μg/ml. The plates were incubated for 72 hr at 37 C, pulsed with 20 μl MTT (5 mg/ml) for 3 hr, and the color estimated spectrophotometrically at 540 nm as described previously. (Schweitzer, C M et al. Spectrophotometric determination of clonogenic capacity of leukemic cells in semisolid microtiter culture systems. Experimental Hematology 21: 573-578, 1993). IC₅₀ values were calculated and compared based on the live/dead ratio of cells. These data teach that all the salts or chelates tested were more effective at inhibiting HL-60 tumor cell growth the free quinic acid (H+). The known chelates such as QA-NH₄+ and QA-Ca++ were more effective than known salts such as QA-Na+. Finally QA-NH₄+ chelate was much more biologically effective than any other salt or chelate, and in effect was as efficacious as the cat's claw water extract (C-Med-100) having quinic acid esters as the bioactive ingredients. These data target chelates of naturally occurring organic acid analogs whether that be in ester or salt form to be chelated with ammonia to increase their efficacy.

For the study of Citrimix as the calcium/potassium alpha hydroxy citric acid chelate compared to the ammonia chelate of hydroxy citric acid (Nu-Citrimix), inhibition growth of primary spleen cells after in vitro exposure in microtiter cell culture for 48 hours in the presence of mitogen (Con A, 2 ug/ml), was the bioassay procedure used (Akesson C., Lindgren H., Pero R. W., Leanderson T., Ivars F., “An extract of Uncaria Tomentosa inhibiting cell division and NF-kB activity without inducing cell death,” International Immunopharm 3:1889-1900, 2003). It was shown that whereas Citrimix IC₂₅ and IC₅₀ values were quite similar to the ammonia chelate (Nu-Citrimix), there was a profound decrease in IC₉₀ values between Citrimix chelate and Nu-Citrimix(ammonium chelate) being 3 mg/ml and 1 mg/ml, respectively. These data were interpreted as demonstrating that Nu-Citrimix(ammonium chelate) had an improved efficacy profile estimated as growth arrest without cell death in primary spleen cells compared to Citrimix, the calcium/potassium chelate of hydroxyl citric acid. Again these data teach the preferred composition for mediating efficacious responses of natural occurring hyroxylated and carboxylated organic acids are ammonia chelates.

### Example II

Example 2 discloses why ammonium chelates of natural occurring polyhyroxylated and polycarboxylated organic acids are the preferred structural analogs for development of nutraceutical or pharmaceutical products to treat health disorders in warm blooded animals. The reasons are theoretically two fold: (i) Ammonium ions are major natural occurring metabolites coming primarily from nitrogen recycling in the gut to balancing and maximizing amino acid and protein biosynthesis that in turn leads to resorption by the body according to its nutritional requirements (Fuller and Reeds, Annu Rev Nutr 18: 385-411, 1998). As such then ammonium ions are important precursors for general support of amino acid and protein nutritional metabolism, and (ii) Ammonium ions are well known to inhibit lysosomal function by neutralizing the acidity inside the lysosome thus preventing oxidative stress radical production, and thereby ammonium ions are important anti-oxidants influencing cellular regulatory processes including immune responsiveness (Seglan, Methods in Enzymology 96: 737-764, 1983).

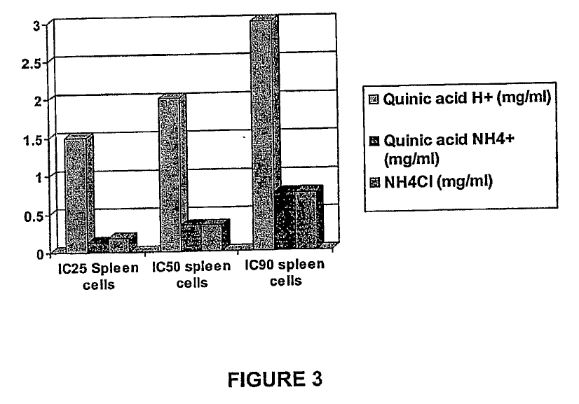

The data presented in FIGS. 3-4 directly support the use of ammonium chelates as a preferred structure for mediating efficacious health benefit of hyroxylated and carboxylated organic acids of natural origin.

It is disclosed in FIG. 3, quinic acid NH₄+ chelate greatly improved the ability of quinic acid (H+) to inhibit growth of spleen cells cultured in vitro by having dramatically lowering the IC₂₅, IC₅₀ and IC₉₀ values compared to quinic acid (H+). Furthermore the data demonstrate that the presence of ammonium ions in the quinic acid N—H₄+ chelate was equally biologically effective because equimolar NH₄Cl by itself had IC₂₅, IC₅₀ and IC₉₀ values comparable to the quinic acid ammonium chelate.

In addition, we have also tested whether NF-kB inhibition is also enhanced by ammonium ions formulated into quinic acid NH₄+ chelate (FIG. 4). Likewise the data were consistent with the fact that quinic acid NH₄+ chelate enhanced NF-kB inhibition over quinic acid (H+) and again the ammonium ions alone in NH₄Cl were as effective as the quinic acid NH₄+ chelate inducing this efficacious response.

In conclusion, quinic acid NH₄+ chelate was shown to have superior in vitro efficacious responses evaluated as growth arrest without cell death and NF-kB inhibition, which in turn are the molecular mechanisms that help enhance immune, DNA repair, anti-inflammatory and anti-tumor properties, already shown by in vivo evaluation of C-Med-100, quinic acid (H+), and ammonia-treated quinic acid (herein structurally elucidated as quinic acid NH₄+ chelate) (Sheng, Y, Akesson, C, Holmgren, K, Brynegelsson, C, Giampapa, V, Pero, R W. An active ingredient of Car's Claw water extracts. Identication and efficacy of quinic acid. Journal of Ethanopharmacology 92: 577-584, 2005; Akesson, C, Lindgren, H, Pero, R W, Leanderson, T, Ivars, F. Quinic acid is a biologically active component of the Uncaria tomentosa extract C-Med 100®. International Immunopharmacology 5: 219-229, 2005).

### Example III

As herein scientifically reviewed, C-Med-100 is an efficacious water extract of cat's claw and it is known to contain quinic acid analogs such as esters. Because both quinic acid and hydroxycitric acid were more biological effective in their ammoniated chelate forms (see Table 3, FIGS. 1-4), then it was reasoned that there may be a manufacturing benefit to formulating quinic acid analogs specifically, and other natural occurring hyroxylated and carboxylated organic acids in plant extracts in general, into their chelated forms by direct treatment of water extracts of cat's claw specifically, and in general other plant extracts, with molecular saturating levels of ammonia. For this purpose, and by way of example, C-Med-100 was first depleted of spray drying agent, maltodextrin, by precipitation with 90% methanol, and then treated with 1% ammonia for 1 hour before being subjected to freeze drying to directly form C-Med-100 ammonia chelate. The excess ammonia was removed by freeze drying. The comparison of C-Med-100 with C-Med-100 ammonia chelate is presented in FIG. 5 as IC values. The data clearly demonstrate an efficacious benefit to ammoniating C-Med-100 in that the IC₅₀ and IC₉₀ values for Nu-CC100 (i.e. ammoniated or nutrated cat's claw having 100% water solubility and thus bioavailability) were more effective at inhibiting the growth of mouse spleen cells than normal C-Med-100. These data were taken as proof of concept that crude plant extracts known to contain hyroxylated and carboxylated organic acid analogs such as quinic acid analogs of unknown structure could be directly placed into ammonia-chelated form thus enhancing their biological effectiveness.

In summation, ammonia treatment of crude plant extracts or bioactives containing carboxy alkyl acid analog structures can be converted into more efficacious formulations by ammonia treatment.

### Example IV

Quin+Cat's Claw Water Extract. Quin+was made in the laboratory by subjecting 75 gm of Uncaria tomentosa bark to 400 ml boiling water extraction for 1 hour. The bark hot water suspension was filtered and centrifuged to remove all particulate matter. Next the extract was evaporated to dryness in a hood with the aid of a hair drier. The yield of solids was 9.9 gm/75 gm or 13.2%. This extract was designated Quin+ and used for all analytical purposes.

### Example V

### Determination that the Quinic Acid Esters in Cats Claw are Quinic Acid Carbohydrate Esters

Glucouronidase experiment. In an effort to distinguish whether the QAE in C-Med-100 were QA esters of carbohydrates their sensitivity to treatment with glucouronidase was examined. C-Med-100 used as commercially supplied was dissolved in water at 710 mg/ml, and then 1 ml left untreated and another ml treated with 30 mg beta glucouronidase (Type B1, bovine liver 1240000 units/gm) for 24 hours at 37 C. Both preparations were then analyzed for breakdown of QA-containing esters by HPLC.

Seliwanoff's test is a colorimetric procedure useful in determining if carbohydrates are present in a sample regardless of whether they may be in monosaccharide, disaccharide, oligosaccharide or polysaccharide forms, or for that matter whether they are esterified or not. Seliwanoff's reagent is 0.05 gm resorcinol in 100 ml 3 M HCl. 0.1 ml of about a 1% carbohydrate is combined with 1 ml of reagent, and the sample boiled for 5-10 min. A deep red precipitate indicates the presence of a ketose sugar such as fructose, whereas a red color developing after more prolonged heating (e.g. 30 min) indicates a hexose sugar like glucose. Four samples were analyzed for carbohydrate content by this procedure. They were D1 R_{f}=0.34, D1 R_{f}=0.56, C-Med-100 and Quin+.

High pressure liquid chromatography (HPLC) analysis. HPLC of water extracts of Cat's Claw bark were carried out using a Perkin Elmer 200 LC pump equipped with a UV detector 785 A. The column was a C18 150×4.6 mm Perkin Elmer-Brownlee (Pecosphere part no. 0258-0169). There was also in tandem but before the 150 mm C18 column a Perkin Elmer C18 30×4.6 mm Brownlee precolumn (P/N N930-3395). The mobile phase that was pumped through the column at 1 ml/min with 1500-5000 psi was either 0.1% trifluoroacetic acid (TFA):methanol (77:23, v/v) or 0.2% TFA:methanol (85:15, v/v). The UV detector was set at the wavelength of 200 nm. An injection loop of 20 μl was used in all experiments. The data were stored and reprocessed using PE Nelson Turbochrom 4 (S270-0052). C18 columns were regenerated following 30 min washes at 1 ml/min with the following sequence of solvents: acetonitrile:methanol (30:70, v/v/), 100% methanol, methanol:water (50:50), methanol:0.2% TFA, and 100% 0.2% TFA.

Next the 1 M NaOH was neutralized with 1 N HCl to pH=4-7. Hydrolyzed samples were between 25-800 mg/ml at the time of HPLC analysis. Identical treated Cats Claw samples except for ± base hydrolysis were compared by HPLC as already described. The QA-H+ generated-base hydrolysis peak eluted from the C18 column in 0.2% TFA:methanol (85:15, v/v) with a retention time of 1.97-2.14 min. Any background peak area appearing in the paired unhyrolzyed sample (i.e. 1.97-2.14 min) was subtracted from the QA calculation as potentially not arising from base hydrolysis. QA concentrations in the hydrolyzed Cats Claw samples were calculated from peak area or peak height according to mV generated after chromatography of standard solutions of QA-H+ from 0-25 mg/ml. No QA could be detected by this method below 3 mg/ml. A linear regression analysis of peak height expressed in mV response to various concentrations of analytical grade QA-+ (Sigma)standard in mg/ml gave a highly significant linear relationship between 3-25 mg/ml calculated as: y=257.7(x)-773, r=0.96.

Direct comparison of +base hydrolyzed samples demonstrated that a quinic acid peak only appeared in the HPLC chromatogram with the after hydrolysis sample, and it was accompanied by several other new peaks representing the carbohydrate moiety of the quinic acid esters present before hydrolysis.

### Example VI

Quinic acid is Vitamin DNA. Vitamins were first discovered in 1929 and won the Nobel Prize that year in physiology and medicine. Now there are 13 well-defined vitamins classified as such because they are essential for life and contribute to good health by regulating metabolism and assisting the biochemical processes that release energy from digested foods. Therefore a “vitamin” is any of the diversified organic compounds required by the body in small amounts (micronutrients), to protect health and for maintaining proper growth in living creatures. 12 of the 13 vitamins (Vitamin D is the exception) cannot be manufactured by the body and so must be derived from the diet in order to maintain optimal health. The U.S. Food and Nutrition Board of the National Research Council recommends dietary allowances (RDA) in order to aid the consumer in identifying and insuring that his health will be adequately maintained with respect to micronutrients.

Already presented here is how important the health of your DNA is to maintaining maximum life's function. In fact it is so important that an entire enzymatic system called DNA repair has evolved to provide a first line of defense of your DNA (genes) to becoming chemically damaged and malfunctioning. The enzymes of this protective mechanism are called endonucleases, exonucleases, polymerases, and ligases. They all have different jobs but they work together to remove harmful lesions in DNA caused by lifestyle, diet and metabolic mistakes 24 hours a day for everyday of your life. The importance of maintaining good DNA repair as a major defense mechanism of the body against the aging process has only just begun to be appreciated in the scientific community during last 20-30 years. DNA is so critical to your well being that DNA repair is now very well known to predict your lifespan. Scientists have repeatedly been able to demonstrate that an animal's longevity is predicted by its ability to carry out DNA repair. For example, rodents live only a couple of years compared to humans who can easily live to 90 years, and humans have 16-times the DNA repair capacity that rodents do. Moreover, there is a linear relationship between mammal longevity in general and DNA repair capacity (Pero, R W et al 1985; Pero et al 2000: Grube, K and Burkle, A 1992). Clearly mammals have evolved into life as we now know it at least in part by protecting the DNA from damage.

Hence, because there are already 13 vitamins existing that are known to help our body maintain good health by catalyzing normal metabolism through dietary intake, then it is only reasonable that the most important metabolic system protecting against aging, DNA repair, would also have a vitamin DNA catalyzed process as well. Here it is presented that a natural product found as a micronutrient in the diet, and not produced in the body called quinic acid is also known to enhance DNA repair, and thus satisfies all the criteria to be classified as a vitamin.

The discovery began in trying to explain the biological activity of a hot water extract of Cat's Claw bark, Uncaria tomentosa, called C-Med-100. This extract was unusually efficient at stimulating DNA repair. After this initial biological characterization of a direct modulation of the single most important DNA protecting process existing in the body; i.e. DNA repair, there has been a concerted effort to identify the natural product behind DNA repair enhancement as a potential anti-aging therapy. A series of chemical studies have led scientists to first identify carboxy alkyl esters (CAEs) as the bioactives and then it was followed by quinic acid esters (QAEs) being identified as the only bioactive CAEs in cat's claw water extracts. Cat's Claw QAEs were bioactive both in vitro and in vivo, however when acid or base hydrolyzed into quinic acid, and without having the alcohol moiety of the QAEs present, quinic acid by itself had as much DNA repair enhancing activity in vivo as did the QAEs. Hence the conclusion that quinic acid was the final biological active form of Cat's Claw, but it was present in water extracts of Cat's Claw as QAES. QAEs are in effect in a pro-metabolite form since the QAEs would be hydrolyzed to quinic acid in the gastrointestinal tract.

On the other hand, quinic acid is quite ubiquitous found in many plants as an essential intermediate in the biosynthesis of most plant aromatic metabolites. Quinic acid cannot be produced by warm blooded animals. So these criteria classify quinic acid as having all the essential properties of a vitamin such as a vitamin DNA. It is not synthesized in the body, but it is present in small amounts in the diet, and can protect the DNA health of individuals against major disease.

Like many vitamins, vitamin DNA, can occur naturally in several forms; namely, as quinic acid esters, quinic acid salts, quinic acid chelates or as free quinic acid in the proton (H+). These various quinic acid natural forms affect modification of the biological activities being mediated by the organism's metabolism at any point in time that can enhance survival. Hippuric acid is the final excretory form of quinic acid (Adamson, R H et al. Biochem J 116: 437-443, 1970) and it likely occurs because quinic acid is a key intermediate in plant benzoylated amino acid biosynthesis (phenylalanine, tryptophan and tyrosine) (Herrmann KM. The Plant Cell 7: 907-919, 1995; Herrmann, K M and Weaver, L M. Annu Rev Plant Physiol Plant Mol Biol 50: 473-503, 1999) including benzoic acid a well known toxicant (Nair, B. Int J Toxicol 20(suppl 3): 23-50, 2001). Hippuric acid is found in high concentrations in the urine of individuals consuming healthy food such as coffee and green/black teas (Mulder, T P et al. Am J Clin Nutr 81(1 Suppl): 256S-260S, 2005), and it has also been reported to act as a hydroxyl radical trap (Malyusz, M et al. Kidney Blood Press Res 24(3) 149-158, 2001). Hence, even hippuric acid may contribute to health effects associated with quinic acid by directly competing with kyrunenine to the inhibit tryptophan IDO (indoleamine 2,3-dioxygenase) degradation pathway (Bauer et al. Transplantation International 18: 95-100, 2004). Thus the P^{H} determines the level of free quinic acid in plant parts and that in turn depends on growth and reproductive factors during plant lifecycle events; for example bearing fruit (i.e. most fruits are acidic and contain much more free quinic acid). However, fruit also requires a high storage of nutritional value to support the initial growing period of the seeds once they start to germinate and growth. This takes time so quinic acid synthesis is then diverted into other forms that help the plants to survive by storage or protection of additional quinic acid food sources such as toxic tannins (Dr Dan Brown, Cornell University, Department of Animal Science, www.ansci.cornell.edu/plants/toxicagents/tannin/) or aromatic esters (e.g. chlorgenic acid) (Clifford, M N. J Sci Food Agri 80: 1033-1043, 2000) or aliphatic esters (e.g. carbohydrate esters in Cat's Claw). Finally, the type of quinic acid salt or chelate forms found naturally are regulated by the soil contents or the intestinal microflora environment where they are growing. Nonetheless, quinic acid whether in the free acid, salt, chelate or ester metabolic forms should all be considered as natural occurring structures of vitamin DNA.

It is not an exception for any vitamin to have several bioactive forms, but rather more like the rule as can be evidenced by review of the B-complex vitamin, niacin. Niacin must be supplied from the diet principally from consumption of grains, nuts, bran, legumes and seeds (Pitche, P T. Sante 15(3): 205-208, 2005). Niacin is metabolized to nicotinamide in the body where both are equally effective as vitamins although niacin causes flushing whereas nicotinamide does not. In addition exogenously supplied L-thrytophan can replace either niacin or nicotinamide vitamin deficiencies (Oduho, G W and Baker, D H. J Nutr 123(12): 2201-2206, 1993). In further parallel to consideration of quinic acid as vitamin DNA, niacin and nicotinamide are metabolized to 1-methylnicotinamide where it is excreted in the urine, and this excretory product also has been shown to have biological activity (Wozniacka, A et al. Clin Exp Dermatol 30(6): 632-635, 2005; Gebicki, J et al. Pol J Pharmacol 55(1): 109-112, 2003).

Thus converting Cat's Claw QAEs to quinic acid or to a more bioactive ester, salt or chelate form, is nothing more than improving the availability of vitamin DNA, now that we are aware of its presence. In this regard Quinmax™ is an optimal formulation for availability of natural vitamin DNA.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

What is claimed is:

1. A process for the production of an isolated medicinal composition for administration to mammals which comprises an effective amount of a free acid, salt or chelate of at least one naturally occurring form of an alpha hydroxyl organic acid comprising:
combining at least one naturally occurring form of an alpha hydroxyl organic acid with an amount of base in an aqueous solution, wherein a total content of alpha hydroxyl organic acids amounts to between about 0.5% and about 35% w/w of the solution, for a time to hydrolyse substantially all forms of alpha hydroxyl organic acids present, neutralizing the solution to a pH between about 6.9 and about 7.6 to yield a free acid, salt or chelate of free acids of substantially all naturally occurring forms of alpha hydroxyl organic acids, and optionally lyophilizing the solution to produce an isolated medicinal composition.

2. A process for the production of an isolated medicinal composition according to claim 1 wherein at least one naturally occurring form of a alpha hydroxyl organic acid is selected from the group consisting of an ester, a carboxy alkyl ester, salt, chelate and a free acid.

3. A process according to claim 2 wherein at least one naturally occurring form of an alpha hydroxyl organic acid is a naturally occurring form of an acid selected from the group consisting of quinic, alpha hydroxyl citric, ascorbic, fumaric, glutaric, lactic, malic, oxalic, tartaric, citric, alpha hydroxy citric, quinic, shikimic, cinnamonic, salicylic, caffeic, hippuric, benzoic, and phenolic acids.

4. A process according to claim 3 wherein the base is selected from the group consisting of NaOH, KOH, and NH4OH and is added to effect a concentration in the aqueous solution within the range of about 0.5M to about 5M for a time between about 15 minutes and about four hours.

5. A process according to claim 4 wherein the naturally occurring form of an alpha hydroxyl organic acid is selected from the group consisting of quinic and alpha hydroxyl citric, and the base is ammonium hydroxide.

6. A process according to claim 5 wherein the solution is neutralized with an acid selected from the group consisting of hydrochloric acid, sulfuric acid, acetic acid, tartaric acid, lactic acid, propionic acid, citric acid, and nitric acid.

7. A process according to claim 6 for the production of an isolated medicinal composition for administration which comprises an effective amount of a quinic acid chelate wherein a ratio of quinic acid to ammonium ion is about 1:1.54.

8. A process according to claim 7 which further comprises the step of combining an effective amount of the lyophilized composition with a pharmaceutically-acceptable carrier suitable for oral administration to a mammal.

9. A process for the production of an isolated medicinal composition which comprises an effective amount of a salt or chelate of a free acid of at least one naturally occurring form of an alpha hydroxyl organic acid comprising:
combining an aqueous extract of plant material, wherein a total content of alpha hydroxyl organic acids in the extract amounts to between about 0.2% and about 35% w/v of the extract, with an amount of base in an aqueous solution for a time to hydrolyse substantially all forms of alpha hydroxyl organic acids present,
neutralizing the solution to a pH between about 6.9 and about 7.6 to yield a free acid, salt or chelate of free acids of substantially all naturally occurring forms of alpha hydroxyl organic acids, and optionally
lyophilizing the solution to produce an isolated medicinal composition.

10. A process according to claim 9 wherein the plant material is selected from the group consisting of uncaria, garcinia, cranberry, and coffee and the base is selected from the group consisting of NaOH, KOH, and NH4OH and is added to effect a concentration in the aqueous solution within the range of about 0.5M to about 5M for a time between about 15 minutes and about four hours.

11. A process according to claim 10 wherein the plant material is uncaria (cat's claw).

12. A process according to claim 11 wherein the isolated medicinal composition comprises an effective amount of a quinic acid chelate wherein a ratio of quinic acid to ammonium ion is about 1:1.54.

13. A process according to claim 12 which further comprises the step of combining an effective amount of the lyophilized composition with a pharmaceutically-acceptable carrier to produce a formulation suitable for administration to a mammal.

14. A process for the production of an isolated medicinal composition which comprises an effective amount of a quinic acid chelate comprising:
combining substantially pure quinic acid, with ammonium hydroxide in an aqueous solution for a time sufficient to reach a pH between about 6.9 and about 7.6, to yield an ammonium chelate of quinic acid wherein a ratio of quinic acid to ammonium ion is about 1:1.54.

15. A process according to claim 14 wherein a solution of ammonium hydroxide, between about 1% and about 10% in concentration, is added to an aqueous solution of quinic acid which comprises between about 5 g to about 30 g quinic acid per 100 ml, in a sufficient amount for the solution to reach a pH between about 7.4 and about 7.6 within a time period between about 15 minutes and about four hours.

17. A process for the production of a functional food which comprises an effective amount of a free acid, salt or chelate of at least one naturally occurring form of an alpha hydroxyl organic acid comprising:
combining a food, which comprises an amount of alpha hydroxyl organic acids in the food between about 0.2% and about 35% w/w, with a base in an aqueous solution for a time to hydrolyse substantially all forms of alpha hydroxyl organic acids in the food,
neutralizing the solution to a pH between about 6.9 and about 7.6 to yield a free acid, salt or chelate of free acids of substantially all naturally occurring forms of alpha hydroxyl organic acids in the food, and optionally lyophilizing the solution to produce an isolated medicinal composition.

18. A process according to claim 17 wherein the food is elected from the group consisting of apple, apricot, garcinia, cranberry, quince, citrus fruits, pineapple, prune, sunflowers, whortleberry, blackberry, red currant, black currant, raspberry, babco, feijoa, kiwano, passion fruit, tamarillo, medlar, persimmon and coffee and the base is selected from the group consisting of NaOH, KOH, and NH4OH.

19. A composition produced by the process of claim 1, 9, 14, or 17 that has been lyophilized and exhibits at least one property selected from the group consisting of: a) inhibits NF-κB activation in Jurkatt T cells to least 50% of the maximum in vitro response at a dose of 1.25 mg/ml or lower, b) causes growth arrest of spleen cells cultured in vitro in the presence of mitogen (Con A) at a dose of 2 mg/ml or lower, and c) systemic administration to a mammal at dose between about 1 mg/kg and 50 mg/kg enhances immune, anti-inflammatory, anti-tumor, DNA repair or tryptophan uptake processes.

20. A method of enhancing DNA repair, enhancing tryptophan uptake, enhancing an immune response, controlling inflammation, or inhibiting the progress of a tumor, comprising administering an effective amount of a composition produced by the process of claim 1, 9, 14, or 17.
